(19) United States
(12) Patent Application Publication
(10) Pub. No.: US 2005/0228595 A1
(43) Pub. Date: Oct. 13, 2005

(54) Processors for multi-dimensional sequence comparisons

(76) Inventors: Laurence H. Cooke, Los Gatos, CA (US); Stephen Eliot Zweig, Los Gatos, CA (US)

Correspondence Address: STEPHEN E. ZWEIG, 224 VISTA DE SIERRA, LOS GATOS, CA, 95030, US

(21) Appl. No.: 11/147,870

(22) Filed: Jun. 8, 2005

Related U.S. Application Data
(63) Continuation-in-part of application No. 10/145,468, filed on May 13, 2002.
(60) Provisional application No. 60/293,682, filed on May 25, 2001.

(51) Int. Cl. G01N 33/48

(52) U.S. Cl. 702/20

(57) ABSTRACT

Improved processors and processing methods are disclosed for high-speed computerized comparison analysis of multiple linear symbol or character sequences, such as biological nucleic acid sequences, protein sequences, or other long linear arrays of characters. These improved processors and processing methods, which are suitable for use with recursive analytical techniques such as the Smith-Waterman algorithm, and the like, are optimized for minimum gate count and maximum clock cycle computing efficiency. This is done by interleaving multiple linear sequence comparison operations per processor, which optimizes use of the processor's resources. In use, a plurality of such processors are embedded in high-density integrated circuit chips, and run synchronously to efficiently analyze long sequences. Such processor designs and methods exceed the performance of currently available designs, and facilitate lossless higher dimensional sequence comparison analysis between three or more linear sequences.

This application is a Continuation in Part of application Ser. No. 10/145,468, filed May 13, 2002, which also claims the priority benefit of provisional patent application 60/293,682 “Processors for rapid sequence comparisons”, filed May 25, 2001.

### BACKGROUND OF THE INVENTION

1. Field of the Invention

The invention relates to improved electronic integrated circuits by which multiple long linear arrays (strings) of characters may be rapidly compared for relationships in a lossless manner. In one application, the linear arrays of characters are biological sequences such as nucleic acid sequences or protein sequences, and the relationships are evolutionary relationships. In another application, the linear arrays of characters are text sequences, and the relationships are closest fit for retrieving appropriately related subject material.

2. Description of the Related Art

The genomes of living organisms typically contain between one and three billion bases of DNA. With each generation, mutations in DNA can occur, and with the progression of time, changes can accumulate to the point where individual genes can change their function. Eventually, this genetic divergence leads to the creation of entirely new species.

Biologists and biomedical researchers often gain considerable insight as to the function of an unknown gene by comparing its sequence with other genes that have similar sequences and known functions. However when more remotely related genes are compared, the DNA sequence that the genes may have originally shared in common may become altered to the point where these relationships are difficult to distinguish from the random background of chance DNA sequence alignment.

Recently, methods for accumulating massive amounts of genetic DNA sequence information have been developed. Because considerable information may be found by comparing the DNA sequences between different genes and organisms, major efforts are underway to sequence the DNA from many different human individuals, and many different species. Due to the high speed of modern sequencing technology, data is now accumulating at a rate vastly in excess of the ability of researchers to interpret it. Thus there is considerable interest in computerized “bioinformatics” methods to automate the difficult process of interpreting this massive amount of data.

Although computer processors designed for general-purpose use have made remarkable improvements in speed and performance in recent years, they are still much too slow for many bioinformatics purposes. For example, to completely compare just two genomes at the simplest level of comparison would require a computer system to construct a two-dimensional matrix, where each side has about 3×10⁹ elements. At a minimum, this matrix would take about 9×10¹⁸ calculations to complete. Assuming each calculation was done in one clock cycle of a typical 1-gigahertz modern computer microprocessor, this matrix would take 9×10⁹ seconds to complete. This is approximately 285 years. If three genomes were compared by this method, the resulting three-dimensional matrix would have 2.7×10²⁷ elements, and would take about 855 billion years to compute.

Because insight into genetic function and relationships plays a key role in modern medical research and drug development, there is a high degree of interest in finding improved electronic devices and methods to speed-up bioinformatic sequence comparisons.

In the area of text searching, the rise of Internet search engines has led to the need for fast searching across large amounts of text data to find the closest fit of portions of this text data to key words or phrases.

Prior Art:

General-purpose computer processors are designed for flexibility, and thus use most of their internal circuitry for general-purpose instructions that are useful for a wide variety of situations. However whenever a more limited set of functions must be done at high frequency, it is often advantageous to employ special purpose logical circuits designed to optimize a more limited set of functions. This is often done for digital signal processors. Such digital signal processors may be optimized to do specialized algorithms for video signals, telecommunications signals, and the like.

To aid in the development of circuits (“chips”) to facilitate high-speed processing of fixed algorithms, field programmable gate array (FPGA) circuits are often used. FPGA circuits are composed of a plurality of simple logical elements, and programmable means to dynamically rewire the connections between the various logical elements to perform different specialized logical tasks. Often this is accomplished by fuse-antifuse circuit elements that connect the various FPGA logical circuits. These fuse-antifuse elements can be reconfigured by applying appropriate electrical energy to the FPGA external connectors, causing the internal FPGA logical elements to be connected in the appropriate manner.

An alternate way to produce custom integrated circuit chips suitable for the implementation of custom algorithms is by more standard chip production techniques, in which a fixed logical circuit is designed into the very production masks used to produce the chip. Because such chips are designed from the beginning to implement a particular algorithm, they often can be run at a higher logic density, or faster speed, than more general-purpose FPGA chips, which by design contain many logical gates that will later prove to be redundant to any particular application.

Often, custom chips are used for parallel processing tasks. Problems amenable to parallel processing may often be subdivided into smaller problems, and each smaller problem assigned to a subunit. If the problem is divided into multiple parts, and each part assigned to a subunit, wherein each subunit performs a logical subpart of the larger problem, it is said to have been divided over multiple processors.

Depending upon the problem at hand, and the means chosen for solution, custom integrated circuit chips can be designed to tackle logical problems by various means. This can range from the entire chip being devoted to one complex and powerful processor circuit containing hundreds of thousands or millions of gates, to the entire chip being devoted to numerous but less capable processors, each individual processor element employing only hundreds or thousands of gates.

Bioinformatics Algorithms:

The typical goal of a bioinformatic genetic sequence comparison algorithm is to report the existence of any statistically significant similarities between two or more genetically different sequences, and to show specifically which elements are similar, and which elements are different.

As evolution progresses, DNA can mutate in many different ways. A DNA base may mutate to a different DNA base by mutation. Alternatively, one or more DNA bases may be either inserted or deleted in a sequence. As evolution progresses, the net sum of many individual mutation, insertion, and deletion changes can potentially reduce the similarity between any two sequences to a very low level. An additional problem rises because the basic genetic code contains only four distinct bases: adenine “A”, thymine “T”, cytosine “C” and guanine “G”. The distribution of these four bases in the genome is non-random, and occasionally, regions will be preferentially enriched in one or more of these bases. Thus even randomly chosen genetic sequences will likely contain some similarities simply due to random base match ups. However such random base matches is of limited biological interest, and usually represents an unwanted statistical “noise” background to all genetic analysis.

Typically, biological sequences, such as DNA, RNA, or protein sequences are stored as linear arrays of characters, where each character corresponds to a DNA, RNA, or protein base, element, or residue. Such linear character arrays are often referred to as “strings”.

The most useful and practical bioinformatics algorithms are those that can find faint but genuine similarities between originally related sequences that have been altered by many mutation, insertion and deletion events, while rejecting random “noise” similarities. Here a number of algorithms have been proposed. Some algorithms, such as BLAST and FASTA, emphasize computational speed at the expense of low-signal-detection ability. Other algorithms, such as the Smith-Waterman algorithm, maximize low-signal-detection ability but are computationally extremely intensive.

In general, if computational means and time permit, the superior low-signal-detection ability of Smith-Waterman like algorithms is preferred because they can uncover faint but statistically valid homologies that faster but less thorough algorithms may miss. Since a new biological insight can potentially lead to discoveries, such as a new drug, that are of significant medical and economic importance, faster methods of performing Smith-Waterman like algorithms are of considerable interest to biomedical research.

The Smith-Waterman algorithm, and related algorithms of this type (such as Needleman-Wunach, etc.) are recursive in nature. They work by considering all possible alignments between two different target sequences, including alignments due to potential insertions and deletions. Their recursive nature is due to the fact that they operate by building upon a history of prior best alignments. In essence they are functions that call themselves over and over. For brevity, discussions here will focus on the Smith-Waterman algorithm as a specific example, however it should be understood that the methods and techniques here could be used with a variety of different comparison functions.

The Smith-Waterman algorithm starts by first considering an alignment between the first elements of the two sequences that it is comparing. It assigns a positive score if the elements (nucleic acid bases) are similar and a zero score if the bases do not match. The algorithm then progresses forward one base and checks if the second elements in the two sequences match (which would occur if the two sequences have homology in this area). If so, another positive score is added to a mathematical matrix cell location that represents the result of this particular comparison. If the two bases do not match, a negative “penalty” score is added to this particular matrix cell location. The algorithm also considers the possibility that one sequence may have had a deletion relative to the other (or alternatively one sequence has an insertion relative to the other). It does this by determining if the second element of one sequence might better match with the first element of the other sequence. If so, this successful match is given a positive score and put into the cell location of the matrix that describes this particular comparison. Because DNA insertions or deletions are less common than simple mutations, such a match is generally a less favored possibility, and is given a lower weight. Thus potential sequence match-ups that would require the assumption that the sequences had undergone many insertion or deletion events are given progressively less weight as the number of potential insertions or deletions grows.

Thus when the two sequences align (are homologous) over a region at a level that is above the random “noise” alignment level, this region of sequence homology will be given a Smith-Waterman score that progressively grows as the number of correct match-ups grows. At the end of the analysis, a two dimensional matrix full of potential Smith-Waterman alignments is constructed. The “best fit” sequence alignment is then found by finding the matrix cell location with the best score, and then tracing back along the chain of previous best fits until the chain ends.

The mathematical form of the Smith-Waterman function is discussed in detail in “Identification of common molecular subsequences” by Smith and Waterman, J. Mol. Biol. 147, 195-197 (1981), the disclosures of which are incorporated herein by reference.

A brief discussion of the algorithm, and an example of its use, are shown below:

Given two sequences, “a” and “b”, with consecutive elements in the sequences represented as “i” for sequence a, and “j” for sequence b, then construct a two dimensional matrix of “a×b”, and populate the matrix according to the algorithm:

| | a1 | a2 | a3 | a4 | a5 |
|---|---|---|---|---|---|
| b1 | | | | | |
| b2 | | 2, 2 | 3, 2 | | |
| b3 | | 2, 3 | 3, 3 | | |
| b4 | | | | | |
| b5 | | | | | |

Here, the contents of matrix cell location SW(i,j) is computed using the formula
SW_{i,j}=max{SW_{i-1,j-1}+s(aᵢ,bⱼ); SW_{i-k,j}+gⱼ; SW_{i,j-k}+gᵢ; 0}

As an example, for cell location [3,3], which represents the results of the comparison between a3 and b3, Swi−1,j−1+s(ai,bi) represents the value passed from the previous best alignment from cell [2,2] (results of comparison a2 vs b2) added to the results from comparing a3 vs b3. If both cell [2,2] and [3,3] contain positive matches, the net result will be the sum of the two, and a larger score number will result. This can be used by the later traceback algorithm to see that good homology can be found between both a2 vs b2 and a3 vs b3.

Suppose however, that there was a gap in one of the sequences, and that [a1, a2, a3, a4, a5] vs [b1, b2, b3, b4, b5] now becomes [a1, a2, a3, a4, a5] vs [b1, new b2 (old b3), new b3 (old b4), new b4 (old b5)]. Then whereas before the deletion, a3 vs b3 would have been a match, now after the deletion, a3 is not a match vs new a3 (old b4). Rather, a3 is now a match vs new b2 (old b3). This potential relationship can be determined by examining the contents of cell [3,2], which shows up in the Smith-Waterman formula as Si,j−k+gj. If this gives a higher match than the other possible combinations, then the possibility that there has been a deletion should be given higher weight.

Here, the “k” elements allow for the consideration that more than one base has been deleted.

In a similar manner, the formula considers deletions in the other string as well through the Si−k,j+gk part of the formula.

The Smith-Waterman formula can be “tuned” by assigning greater or lesser coefficients to matches vs non-matches. It can also be tuned by assigning greater or lesser coefficients to deletion and insertion events. Since such deletions or insertions are generally less common, these are usually given negative weighting factors.

After the matrix is computed, the maximum cell location is used as the starting point of the analysis, and the maximum values are then traced back until 0 is reached. This traced back matrix then corresponds to the region of maximum homology between the two sequences.

It should be noted that this calculation is recursive in nature. That is, the algorithm works by calling itself numerous times. As a result, it is very computationally intensive to perform this calculation on standard computers. Nonetheless, due to its high ability to find biologically interesting homologies between biological sequences, this algorithm is widely used in bioinformatics

Due to computational limitations, essentially all lossless comparison prior art (that is, prior art that does not substantially rely upon lossy compression of nucleic acid sequence information) has focused on comparing only two sequences at a time, (using two-dimensional Smith-Waterman analysis, or other type of 2 D analysis). However biologists are well aware of the importance of studying the relationships between three or more different sequences. Studying the relationships between many related genes allows researchers to determine similarities and differences between genes, and make inferences as to the function of new genes. As discussed in chapter 8 “Practical Aspects of Multiple Sequence Alignment”, by A. Braxevanis (published in “Bioinformatics, A practical guide to the analysis of genes and proteins” by Baxevanis and Ouellette, Wiley Inter-Science, New York, 1998), a number of computerized methods of multiple sequence alignment are known in the art. All, however, rely upon first performing multiple 2 D analysis to first discover any relationships, and then summing the results of many different 2 D analysis using logic such as: “if A is related to B, and B is related to C, then A must be related to C even if a direct 2 D analysis of A versus C shows no detectable homology. Such analysis by necessity is a lossy comparison that does not utilize the full information contained in the original data set, and thus may miss important homologies. Examples of such prior art for multiple sequence alignment include programs incorporating algorithms such as “CLUSTAL W”, MULTIALIN, BLOCKS, MOST, PROBE, and the like.

Although, possibly due to computational limitations, there appears to be no prior art teaching the desirability or methods of doing lossless higher dimensional sequence comparisons between three or more target sequences with unknown homology, such comparisons would appear to be desirable. This is because of the nature of biological DNA sequence divergence. A single ancestral DNA sequence will typically, after genetic divergence into different organisms, undergo mutations, insertions, or deletions in different places. By making use of consensus logic (“two out of three” algorithms, etc.), faint low-homology matches between distantly related DNA sequences may be discovered that might otherwise be missed using conventional multiple 2 D approaches. For example, consider four sequences. If 2 D analysis can demonstrate that sequences A, B, and C are each homologous to sequence D, yet fail to detect any homology when A, B, and C are each compared to one another, then the relationship between A, B and C will not be discovered by 2 D analysis if sequence D is not available. By contrast, a more comprehensive 3 D analysis may uncover the relationship between sequences A, B and C even when sequence D is unavailable.

As previously discussed, even two-dimensional Smith-Waterman type analysis tax current general-purpose computer systems past their limits. Thus there have been prior attempts have to implement custom integrated circuit chip systems specifically designed to speed-up this type of bioinformatic algorithms.

The simplest method to speed up calculations of this nature is to use more computer processors. The dot matrix comparison programs of Zweig “Analysis of large nucleic acid dot matrices on small computers”. (Nucleic Acids Res. 1984 Jan 11; 12(1 Pt 2):767-76) were designed to allow larger sequence comparisons to be broken up into smaller chunks and run on multiple unnetworked computers. Other early work in this field includes Collins and Colson, “Applications of parallel processing algorithms for DNA sequence analysis” (Nucleic Acids Res. 1984 Jan. 11; 12(1 Pt 1): 181-92), as well as Bernstein “Using spreadsheet languages to understand sequence analysis algorithms” (Comput Appl Biosci 1987 September; 3(3):217-21).

In addition to multiple unnetworked computers, calculations may also be done by dividing the problem up and passing parts of the calculation to networked computer processors. This network of computer processors can be a specialized multi-processor computer, as discussed in “Supercomputers and biological sequence comparison algorithms” by Core et. al., (Comput Biomed Res 1989 December; 22(6):497-515). Alternatively networks of conventional computers can also be used for this purpose, as discussed in Barton “Scanning protein sequence databanks using a distributed processing workstation network”, (Comput Appl Biosci 1991 January; 7(1):85-8).

To proceed still further, one obvious solution is to embed many general-purpose microprocessor circuits onto one chip, and run these general-purpose microprocessors simultaneously. However since general-purpose microprocessors are typically not optimized to perform any specific computational task, this solution is inefficient because a general-purpose microprocessor typically contains extra circuitry that is unneeded in any specific implementation. Further, general-purpose microprocessors typically require more clock cycles to perform a given computational task than a computational circuit dedicated to a specific problem.

As a result, there has been interest in designing computational circuits optimized for specific computational problems, and then embedding a plurality of such optimized circuits into integrated circuit chips in order to address complex problems by many simultaneous computations. There are a number of prior examples of this type of approach.

U.S. Pat. No. 5,168,499 teaches fault detection methods to produce reliable operation of multiple instances of a specific type of processor on a VLSI sequence analysis chip.

U.S. Pat. No. 5,632,041 (Peterson) and U.S. Pat. No. 5,964,860 teach a 400,000 transistor, 100,000-gate custom VLSI chip composed of sixteen instances of a specific type of processor design, described in FIGS. 11, 12 and 13 of that patent. These patents teach a type of specialized processor design in which each processor is partially optimized to do Smith-Waterman type calculations. However the design still requires a fair number of clock cycles per Smith-Waterman cell calculation. The efficiency of the '041 “Peterson” design is limited because it lacks the circuitry and support registers necessary to execute multiple comparisons on a per-processor and per-clock cycle basis.

U.S. Pat. No. 5,873,052 teaches computer processing methods optimized for distinguishing between different nucleic acid sequences that have been previously identified as being highly related.

It is evident that ganging up many custom chips (each employing a plurality of processors) into a larger computational system will further enhance processing speed. Here a number of methods may be used. In addition to the methods taught by the '041 and '860 patents, U.S. Pat. No. 6,112,288, teaches alternative pipeline methods useful for configuring arrays of multiple processor chips into a larger overall system.

Other alternative methods of performing sequence analysis, previously known in the art, include “content addressed memory techniques”. For example, U.S. Pat. No. 5,329,405 teaches content addressed memory circuitry methods for finding regions of exact sequence matches between two given sequences. However such methods are not well suited to perform high-sensitivity comparisons where the various sequences may have multiple mutations, insertions, or deletions.

Commercial embodiments of the prior art can be found in systems by Paracel and TimeLogic. For example, Paracel Corporation (Pasadena, Calif.) produces the GeneMatcher2™ genetic analysis system, based upon custom VLSI sequence analysis chips. TimeLogic Corporation (Incline Village, Nev.) produces the DeCypher™ genetic analysis system, based upon custom FPGA chip technology.

Hirschberg et. al. (KESTREL: A Programmable Array for Sequence Analysis. in IEEE Computer Society Press, ASAP'96, Chicago, Ill., August 1996, pp: 25-34) disclose Kestrel, a programmable array for sequence analysis. Kestrel is a programmable linear systolic array processor designed for sequence analysis. Although composed of multiple processors, each of Kestrel's processors is non-pipelined, and each operates on a one instruction per clock cycle basis. As a result, Kestrel's processors lack the additional registers required to perform independent interleaved computations per clock cycle. Moreover, Kestrel does not contemplate comparisons between three or more sequences. As a result, Kestrel does not teach consensus functions, which are required for comparisons between three or more sequences.

Borah et. al. (M. Borah et. al. “A SIMD solution to the sequence comparison problem on the MGAP,” in ASAP, IEEE CS, August 1994) discloses a Micro-Grain Arithmetic Processor. According to Borah, “The Micro-Grain Arithmetic Processor (MGAP) has its own reconfigurable architecture with a 64×64 mesh of sequence comparison cells on the original chips and a 128×128 mesh on the next-generation MGAP-2. The mapping works on 128 128-long comparisons at once—a linear pipeline would have somewhat lower total MCUPS but could handle an isolated query longer than 128 characters”. (Borah's MGAP work was also quoted by Hughley et. al. in his disclosure of Kestrel via Hirschberg et. al.) By configuring an N×M two-dimensional array of MGAP processors Borah is able to perform K comparisons between K different N length strings and one M length string in M+N+K steps.

Borah's system accepts only two nucleic acid sequences as input data, and fails to teach consensus functions. Although Borah suggests the need for multiple comparisons, the purpose of these is to break down the analysis of two arrays of input data into smaller and more manageable chunks. Borah uses a two dimensional array of processors to perform these multiple comparisons, and because of the fine-grained nature of the MGAP processor, takes multiple clock cycles to perform each step.

Hughley, (“Parallel hardware for sequence comparison and alignment, CABIOS 12(6), 473-479, Oxford University Press, 1996) reviews a number of different sequence analysis coprocessors. This paper discusses the problems of using the computationally intensive “fine grain” sequence comparison algorithms, such as the Smith-Waterman algorithm, for very large nucleic acid databases for anything other than special purpose processors. The article reviews a number of “shortcut” (lossy or coarse grain) algorithms that allow large databases to be quickly if less thoroughly addressed with a lower computational load, and do not require special purpose processors. The article also reviews a number of prior Smith-Waterman special purpose processors such as the Time Logic approach and Kestrel approaches discussed previously.

Alternative “shortcut” homology algorithms, such as hidden Markov methods, are frequently used in nucleic acid analysis because, as recently stated by Lyotyonja and Milinkovitch, “Exact inference of optimal multiple sequence alignment is computationally impractical for more than a few short sequences” (“A hidden Markov model for progressive multiple alignment”, BIOINFORMATICS Vol. 19 no. 12 2003, pages 1505-1513). In this context, “exact inference” means lossless methods, such as the Smith-Waterman method, and earlier versions of this approach such as the Needleman and Wunsch algorithm (J. M. Biol. (1970) 48, 443-453).

Hidden Markov models analyze stretches of nucleic acids, compress the large amount of information contained in the stretch to a smaller amount of information that roughly but not exactly describes the larger stretch (lossy compression), and then speeds up homology determination by doing comparison operations using lossy compressed data. The approach is similar to that of trying to identify a suspect by matching his face in a database. If the suspect has blonde hair, then the search can be made faster by first selecting for all blond hair individuals in the database.

Lossy comparison methods, such as hidden Markov models, are indeed quicker than lossless homology algorithms (such as the Smith-Waterman type algorithm), but by necessity sometimes miss potential match-ups. What if the suspect has dyed his hair? A lossy comparison that relies on hair color may fail to identify the suspect, while a more complete lossless comparison of all identifying features may find the suspect.

Hidden Markov models are also useful for analyzing protein sequences. Since each amino acid in a protein is specified by a 3-base nucleic acid codon, and since multiple different 3-base codons code for the same amino acids, then there are hundreds or thousands of alternative nucleic acid sequences that can specify the same amino acid chain. Two different proteins may superficially not have an obvious sequence homology, but consideration of amino acid mutation paths and codon substitution may reveal a hidden relationship. Here hidden Markov models, such as those discussed by Hughley, and by Eddy et. al., (Maximum discrimination hidden Markov models of sequence consensus. J Comput Biol. 1995 Spring; 2(1):9-23) can help address this issue, and can allow sequences to be compared on more of a pattern recognition basis, rather than on the Smith-Waterman type basis described in this disclosure.

So far, no processors optimized for executing the vastly more complex hidden Markov nucleic acid comparisons in a small number of clock cycles have been described in the literature. Rather, hidden Markov nucleic acid comparisons are currently done by general-purpose computer microprocessors that execute hidden Markov programs written in conventional programming languages.

Although prior art has attempted to facilitate Smith-Waterman analysis by a variety of specialized processor techniques, such systems still cannot obtain the speed needed to perform many biologically interesting analyses. Additionally, no-doubt due to processing speed limitations, there appears to be no prior art whatsoever on special purpose electronic systems optimized for performing multi-dimensional Smith-Waterman type analysis. That is, all prior art special purpose processors that are capable of performing Smith-Waterman type analysis in a small (less than 3 to 10) number of clock cycles are capable of accepting only two arrays (strings, dimensions) of nucleic acid input data, and can output only two coordinates (the position in the first array, and the position in the second array) when significant matches between the two arrays are found. Thus the need for improved equipment and methods of computer sequence analysis is apparent.

### SUMMARY OF THE INVENTION

In order to obtain greater insight into many biological processes, an enhanced ability to simultaneously compare three or more related nucleic acid sequences, such as nucleic acid sequences obtained from different species of animals, is highly desirable. This is computationally challenging, and requires specialized processors in order to proceed at a reasonable rate of speed. Previous specialized processors, however, were limited to only two-dimensional comparisons, and also did not fully utilize pipelining techniques to maximize speed.

The present invention discloses a specialized processor that is capable of simultaneously analyzing three or more nucleic acid sequences. Moreover, the present invention discloses the utility of using pipelining techniques to perform multiple operations during a single clock cycle, which can be used to speed up the large number of calculations required for higher dimensional analysis.

Since, as previously discussed, it would take approximately 855 billion years to analyze three human-genome-sized sequences using conventional microprocessors, the need to improve computational efficiency should be apparent. The first part of this disclosure discusses how such computational efficiencies can be obtained, and the second part of this disclosure discusses how practical three-dimensional processors may be constructed.

In order to appreciate how the present invention differs from prior art, and why these differences are advantageous, a discussion of the relative computational efficiencies of the various prior art techniques is in order.

Previous workers have attempted to implement flexible custom sequence analysis integrated circuit chips composed of either multiple processor units on one chip, or alternatively using dynamically reconfigurable logical elements using FPGA technology. However such flexibility comes at a cost. Processor elements must contain additional gate elements and/or operate over many clock cycles in order to store and retrieve instructions and data elements from registers, and thus tend to be gate and clock cycle inefficient. Field programmable logical circuits must, by necessity, contain many redundant gates that are unused in any particular programmed logical application, and thus are also quite gate inefficient. In either event, for any given chip design with a given number of gates and clock speed, these inefficiencies dramatically reduce the maximum potential computational speed, or the size of the strings that can be compared

The performance damaging effect of gate inefficient or clock cycle inefficient designs can be readily appreciated. The ideal circuit design will calculate as many Smith-Waterman cell locations as possible per integrated circuit chip and per clock cycle. Typically one processor will be responsible for updating the contents of one Smith-Waterman matrix location cell. Under these conditions, if all cells update with the same number of clock cycles, then an integrated circuit with 400,000 logical gates divided into 10 40,000 gate-sized Smith-Waterman processors will operate at 10× lower efficiency than a 400,000 logical gate chip divided into 100, 4,000 gate-sized Smith-Waterman processors. Similarly, a 100 MHz integrated circuit chip running with processors that require 40 clock cycles per Smith-Waterman cell calculation will operate at 10× lower efficiency per processor than a 100 MHz integrated circuit chip running with processor units that require only 4 clock cycles per Smith-Waterman cell calculation.

We have found that using processor units with both low gate counts, and good clock cycle efficiency, efficiencies of nearly 25-100× may be achieved over alternative designs. This gain in processing capability in turn enables more sophisticated types of genetic analysis, hitherto infeasible on conventional computational systems.

As previously discussed, FPGA circuits for genetic analysis are taught in the commercially produced DeCypher system, produced by TimeLogic Corporation, Incline village, Nev. According to TimeLogic's most recent (2001) product literature, the most recent embodiment of this prior art system incorporates six logical Smith-Waterman processors per 250,000 gate Altera Flex 10 K FPGA chip (EPF10K250A), and operates at an effective calculation speed of 52,000,000 Smith-Waterman cell updates per second per processing element subunit. Each processor requires approximately 42,000 FPGA gates. The processor efficiency, in terms of Smith-Waterman cell updates per logical gate per second, is about 52,000,000/42,000 or about 1250 Smith-Waterman cells per logical gate per second.

By contrast, the VLSI circuit multiple processor approach is taught in the commercially produced GeneMatcher2 system, produced by Paracel Corporation, Pasadena, Calif. According to the most recent product literature (2001), the most recent embodiment of this prior art system incorporates 192 logical Smith-Waterman calculating subunits per 30,000,000-transistor VLSI chip. Each processor requires 39,000 gates, and each processor operates at a calculation speed of 10,000,000 cell updates per second. The efficiency, in terms of cell updates per logical gate per second is about 10,000,000/39,000=256 Smith-Waterman cells per logical gate per second.

Although different custom processors have different efficiencies, all custom processors exhibit Smith-Waterman calculation efficiencies that vastly exceed those of conventional, general-purpose processors. A comparison to general-purpose microprocessors can be found in the work of Rognes and Seeberg in “Six-fold speed-up of Smith-Waterman sequence database searches using parallel processing on common microprocessors”. Bioinformatics 2000 August; 16(8):699-706. Here, they report on a highly optimized parallel Smith-Waterman algorithm running on a conventional Pentium II processor at 500 MHz. This achieved 150,000,000 cell updates per second. The Pentium III chip contains one computing unit, with approximately 28,000,000 transistors (7 million gates). Thus the efficiency is 150,000,000/7,000,000 or about 21 Smith-Waterman cells per logical gate per second, vastly lower than even the slowest custom processors.

By contrast, systems using the concepts taught in this invention, 2 dimensional Smith-Waterman calculating processor units can be implemented at approximately 6,500 gates per subunit, and can operate at over four times the clock speed as the DeCypher FPGA system (approximately 187,000,000 cell updates per second). The efficiency, in terms of cell updates per logical gate per second is over 28,000 Smith-Waterman cells per logical gate per second. Thus for equal integrated circuit gate counts and clock speeds, the design of this invention produces calculation efficiencies about 20-40 times higher than prior art.

In the first aspect of the invention, improved interleaving methods to construct efficient Smith-Waterman type processor calculating circuits, suitable for nucleic acid analysis, protein analysis, or general character similarity analysis between two strings, with extentions to handle two independent comparisons in parallel are disclosed

In a second aspect of the invention, methods for doing higher dimensional comparison analysis between three or more input strings (genetic sequences) are taught.

In a third aspect of the invention, integrated circuit chips containing multiple hardwired complex logical circuits that accomplish higher dimensional comparison analysis between three or more input strings (genetic sequences) are disclosed.

Definitions:

Homology: “homology” means the extent to which the two or more nucleic acid sequences, amino acid sequences, one-dimensional character arrays, or text strings (i.e. sequence of text characters or a text file) are similar or are related to each other. If they are similar, then for some positional length along each of the various respective one dimensional arrays or sequences, there will be regions where the characters (nucleic acid bases, etc.) from the various sequences match-up at a higher than random chance level.

For the purposes of this discussion, “homology” can also be mathematically described by the “similarity algorithm” as defined by Smith and Waterman.

Typically, numerical homology scores are produced by comparing the two or more sequences (i.e. the input content within the two arrays), and incrementing a counter whenever the specific sequence elements (nucleic acid bases in the case of nucleic acids, amino acid sequences in the case of proteins, or specific text characters in the case of text files) between the two or more sequences are found to be the same. Two or more sequences that are very similar to each other will have many such counter increments, and thus will have a “high homology”. By contrast, in the case where the two or more sequences are dissimilar, the counter will increment much less often, and a “low homology” score will result.

Note that the starting index (array location) where a similar region exists may not have the same location on a first array (sequence, string) as it does on a second array. For example, consider the two sequences:
Sequence 1: JHFGLHOMOLOGYIUIUTY
Sequence 2: YTHOMOLOGYYETJFKJG

It is clear that the homologous region between sequence 1 and 2 begins at character 6 on sequence 1, and on character 3 on sequence 2. Thus “homology” would begin at coordinates (6,3) in this example. As alternate phrasing, the “coordinates where maximum homology occurs” would correspond to this location (6,3). As yet another alternate phrasing, the numbers “6” and “3” could also be designated as “pointers” to the regions of highest comparison between the input strings.

Consensus: “consensus” means general but not necessarily unanimous or perfect agreement. In the case of two-dimensional analysis, establishing “consensus” between any two-sequence elements (or one-dimensional array characters) is rather trivial—the two characters either agree or they do not. However, as the present invention discloses, the concept of “consensus” is highly meaningful for higher dimensional comparisons.

In the context of comparing three or more sequence elements (nucleic acid base, amino acid residue, or character), consensus means the process of determining if there is a unanimous agreement (for example 3 out of 3 sequence match), an imperfect agreement (for example a 2 out of 3 sequence match), or a total lack of an agreement (all three sequences don't match). Typically a consensus function will return a higher value if the amount of agreement is higher, and a lower value if the agreement is lower. Such a consensus function is thus one of “partial credit and greater credit”.

Note that consensus functions may be used to control the counter in a homology function (discussed above). In this context, a consensus function is a function that operates on three or more “input arrays”, i.e. input sequences (nucleic acid sequences, amino acid sequences, or text files), selects specific “input characters” or “input elements” from these input sequences (input arrays), where these “input characters” can be specific nucleic acid residues, specific amino acid residues, or specific characters from a text file, and determines the extent of consensus among the specific input elements that were chosen.

Array, “sequence” and “string”: “array” is used in the common programming or mathematical sense as a synonym for “sequence”. Unless otherwise stated, the term “array” is considered to be a linear (one-dimensional) array of characters. For example, a nucleic acid sequence can alternatively be described as a (one dimensional) nucleic acid array, or a text file can be alternatively described as a (one dimensional) character array. “String” is also used in the common programming sense as meaning a one-dimensional array or one-dimensional sequence of characters. These characters can be text characters, nucleic acid residues, or amino acid residues. Every character or element in a string, sequence, or one-dimensional array has a unique coordinate or location that can be described by a single integer. For example, given the one-dimensional array, string, or sequence “GAAATTDAATT”, if the location or coordinate of “G” is defined to be 1, then the location of “D” would be 7.

Characters: The individual elements (characters) in a one-dimensional array are typically represented in computerized devices by 7 or 8 bit ASCII characters. However when nucleic acids are represented, the four base alternates (A, T, G, C) can be represented with only two-bits of information, which can simplify comparison hardware and allow for greater memory efficiency. Alternatively, for more complex text comparison applications, each character (individual array element) may be represented by 16, 21, or 32 bit Unicode characters.

Pipelining: “pipelining” is a specific way of performing multiple operations at the same time. For example, successive staging of multiple computations such that at least one completes after one or more has started. In this technique, a microprocessor or other electronic circuit capable of following a list of instructions begins executing a second instruction before the first has been completed. That is, several instructions are in the being executed by the microprocessor or electronic circuit simultaneously, each at a different processing stage. Such pipelining enables a microprocessor or other electronic circuit to “execute a first portion” of the problem at hand while also “executing a second portion” of the problem at hand.

Interleaving: “interleaving” is doing similar operations on out of order data in an alternating or rotating fashion. For example interleaving pixels on a TV, means two rows are sent in an offset parallel arrangement, alternating between the rows for each pixel. In our context, interleaving means alternating independent calculations in a pipeline.

Folding: “folding”—in our context, when the interleaved data is another section of one (or more) of the strings being compared.

Loaded: in our context this is data, which is loaded into registers prior to beginning the comparison process.

Input: Input data is defined to be raw data, such as experimentally determined nucleic acid sequence data, or unprocessed text data, that exists in a fixed form prior to the start of the comparison process, and has not had information removed by any lossy compression method. By contrast, the many ways in which a string of data (character array, nucleic acid sequence, etc.) can be chopped or modified after the comparison process begins is not considered to be input data, but is rather considered to be an intermediate calculation step. Thus a processor that accepts only two arrays (strings, nucleic acid sequences) of lossless data as input, but then produces many chopped, compressed, or modified forms of the two arrays as an intermediate calculation step, would be considered to be performing only a two dimensional analysis, rather than a three dimensional or higher dimensional analysis. Thus a good test to determine the true dimensionality of a processor is to examine the number of lossless input data sequences (strings, character arrays, etc.) that the processor accepts.

Inputted: in our context this is data, which is inputted, one character at a time, during the comparison process.

Lossy: a method of handling data or data compression in which some data is lost

Lossless: a method of handling data or data compression in which no data is lost.

### BRIEF DESCRIPTION OF THE DRAWINGS

FIG. 1 illustrates the inefficiencies of the prior art, by showing the order in which the processor components are utilized serially over one clock cycle, thus limiting the speed of the processor.

FIG. 2 shows an improved processor; designed to perform one cell of a Smith-Waterman calculation may be constructed following the teachings herein.

FIG. 3 shows how multiple processors from FIG. 2 may be interlinked on an integrated circuit chip capable of performing a multi-element Smith-Waterman analysis, following the teachings of prior art.

FIG. 4 shows a processor of the present invention that utilizes interleaving techniques to improve efficiency.

FIG. 5 shows how multiple processors of the present invention may be interlinked on an integrated circuit chip capable of performing single or multiple multi-element Smith-Waterman analysis.

FIG. 6 shows some of the efficiency improvements that may be gained by following the teaching taught herein. Here, the order in which the improved processor elements are utilized over multiple clock cycles is shown.

FIG. 7 shows a conceptual diagram of how higher dimensional Smith-Waterman like analysis may be performed. Here, a three dimensional analysis with three different input strings (genetic sequences) is shown.

FIG. 8 shows a top-level diagram of a three-dimensional processor of the present invention, along with diagrams of each of the components shown at the top level.

FIG. 9 shows how multiple three-dimensional processors of the present invention may be interlinked on an integrated circuit chip capable of performing a multi-element three-dimensional Smith-Waterman type analysis.

FIG. 10 shows a three-dimensional processor of the present invention that utilizes interleaving techniques to improve efficiency, in the same fashion as FIG. 4.

FIG. 11 shows the details of the functions shown in FIG. 10.

FIG. 12 shows how multiple processors of the present invention may be interlinked on an integrated circuit chip capable of performing a multi-element, three-dimensional Smith-Waterman analysis.

FIG. 13 is an abstraction of two-dimensional non-interleaved and interleaved functions.

FIG. 14 is an abstraction of three-dimensional non-interleaved and interleaved functions.

FIG. 15 shows a top-level diagram of an N-dimensional processor of the present invention.

FIG. 16 is a diagram of one of the components shown at the top level in FIG. 15.

FIG. 17 is a diagram of the order in which N dimensional processor elements are utilized over multiple clock cycles when processing three independent comparisons in parallel.

FIG. 18 is a diagram of the order in which N dimensional processor elements are utilized over multiple clock cycles when processing four independent comparisons in parallel.

### DETAILED DESCRIPTION OF THE INVENTION

Prior art processors contain a variety of subcomponents (adders, comparators, registers, and the like) and require a number of clock cycles to complete an analysis. Because the analysis requires multiple clock cycles, only a fraction of the processors' subcomponents are used during each clock cycle. Those processor components that are unused in any given clock cycle represent wasted resources. If these components are not fully utilized, then efficiency can be improved by using fewer components per processor, and putting more processors on a chip; redesigning the processor to more fully utilize all components by pipelining the operations, or a combination of the two.

We have found that by employing novel and improved processor designs that make more efficient use of processor subcomponents, significant improvements in calculating efficiency over the prior art are possible. Large-scale bioinfomatic systems of the prior art typically can cost from millions to hundreds of millions of dollars to implement. These systems, in turn, are used to generate leads for new drug discovery efforts. Since new drugs can produce revenues the billion-dollar range, the economic benefits of improved processor design (lowered computing costs, more drug leads) should be evident.

By use of these improved designs, a larger number of processor units may be placed on integrated circuit chips, and correspondingly faster processing may be done. Use of the improved processor units also enables practical circuits to be constructed that, for the first time, enable higher dimensional (three dimensions or more) sequence analysis to be a practical analytical option.

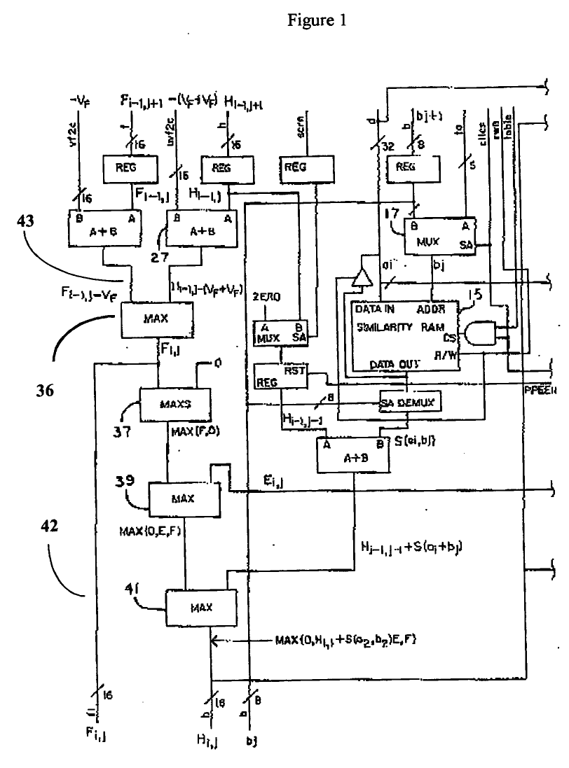

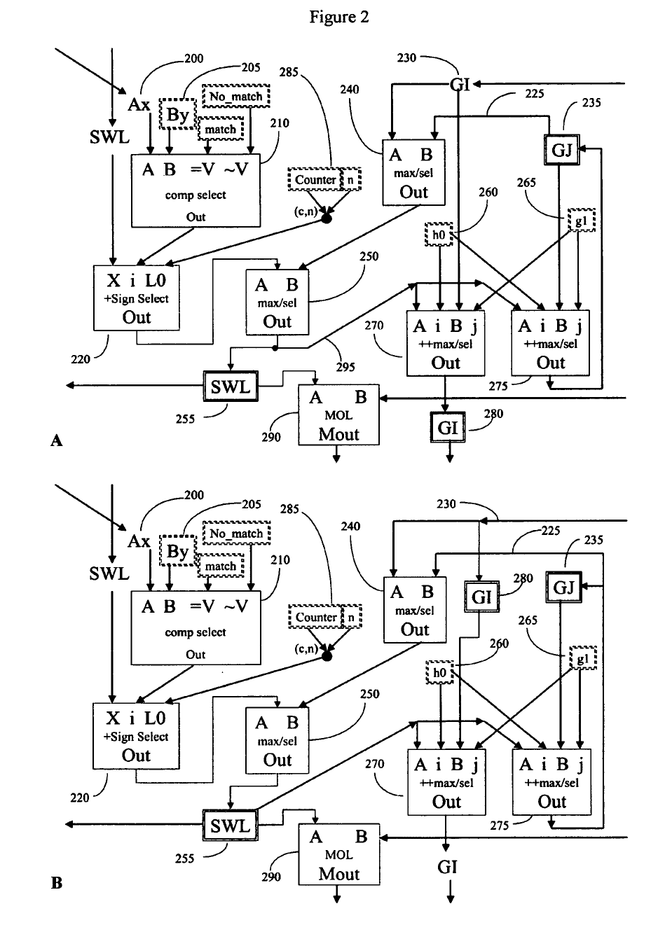

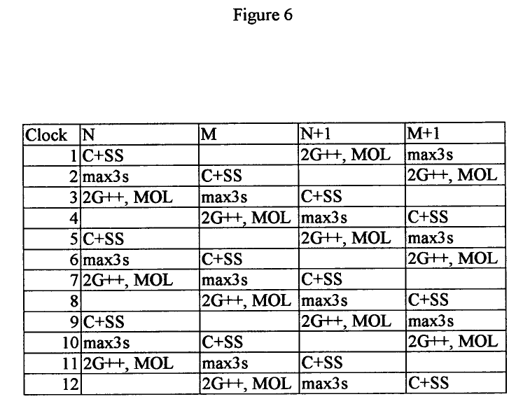

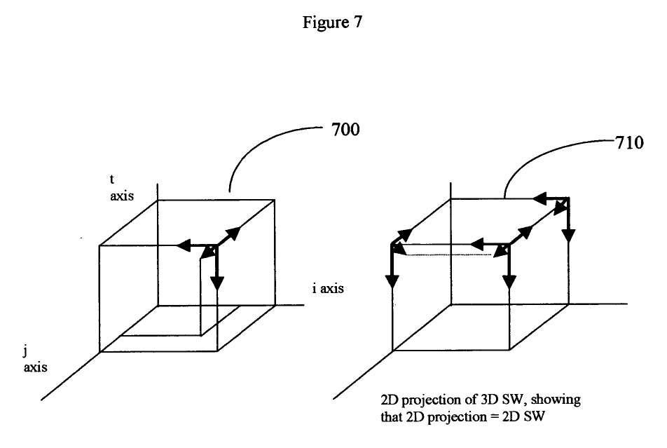

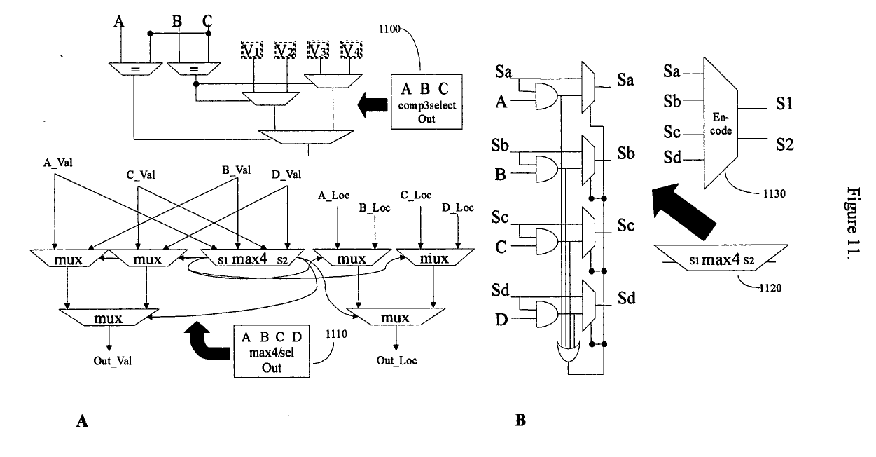

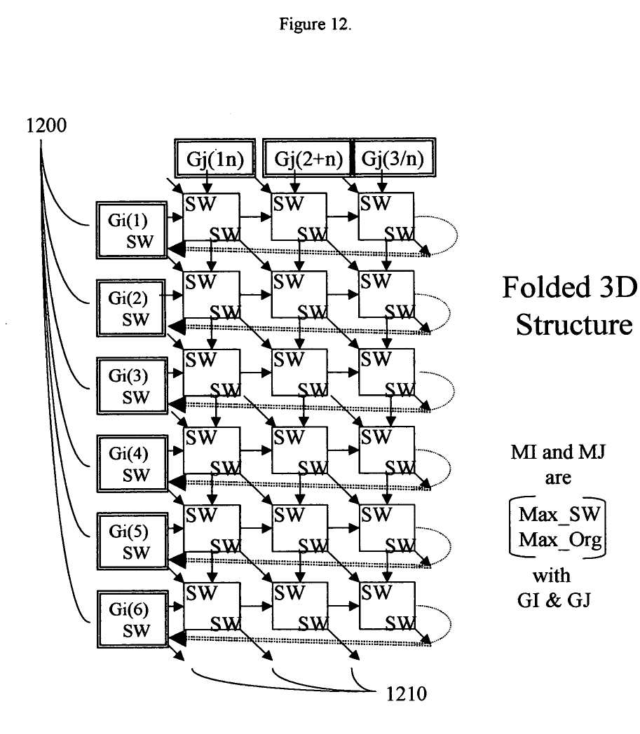

FIG. 1 shows the order in which the various processor components are used following the teachings of prior art, taught in FIGS. 11 and 12 of U.S. Pat. Nos. 5,632,041 and 5,964,860. Note that many processor components are idle for most of the clock cycles during the completion of one Smith-Waterman cell analysis. During the clock cycles from the start of the operation, and the computation of the final Smith-Waterman value, most of the subunits are used only a small fraction of the time. Typically many subunits are idling while either waiting their turn, or after their turn. Note that the path in FIG. 1 from Hi−1,j+1 at the top, down to Hi,j at the bottom, is used to calculate the completion of one Smith-Waterman cell analysis. Unfortunately the signals must propagate through an adder [A+B] (27), three Max functions (36, 39, and 41) and one Maxs function (37). By comparison in FIG. 2a, the SWL value goes through two adders three max functions and one Maxs function, which is no better than the prior art, but by rearranging the order of the computation as shown in FIG. 2b, the critical path is either through an adder, a max function and a maxs function, or through an adder and two max function, both of which are less delay than the prior art It should be noted here, that while prior art such as that shown in FIG. 1 occasionally delineates the number of signals or width associated with a line with a number and slash through the line as in (42), other signal line widths are not defined such as (43), and are left to the reader to assume. In this patent the reader should assume all signal line widths in all of the subsequent FIGS. 2 through 16, except FIGS. 6 and 7, to consist of multiple bit lines. The actual widths will vary and should be defined by the size of the problem, and therefore the size of the numeric calculations required, but should at least be 16 bits wide, and preferably 32 bits wide or larger.

FIG. 2a is a rearrangement of the organization of the computation. Starting with the basic Smith-Waterman equation:
SW_{i,j}=max{SW_{i-1,j-1}+s(aᵢ,bⱼ); SW_{i-k,j}+gⱼ; SW_{i,j-k}+gᵢ; 0} for all k<i or j

If we assume that gᵢ and gⱼ are of the form g₀+k*g₁ the middle terms have the form:
SW_{i-k,j}+gⱼ=max{SW_{i-1,j}+g₀+g₁; . . . SW_{i-k,j}+g₀+k*g₁; . . . SW_{1,j}+g₀+i*g₁}
And
SW_{i,j-k}+gᵢ=max{SW_{i,j-1}+g₀+g₁; . . . SW_{i,j-k}+g₀+k*g₁; . . . SW_{i,1}+g₀+j*g₁}

Which is equivalent to:
SW_{i-k,j}+gⱼ=max{SW_{i-1,j}+g₀+g₁; g₁+max{SW_{i-k,j}+g₀+k*g₁; . . . g1+SW_{1,j}+g₀+j*g₁} . . . }
And
SW_{i,j-k}+gᵢ=max{SW_{i,j-1}+g₀+g₁; g₁+max{SW_{i,j-k}+g₀+k*g₁; . . . g1+SW_{i,1}+g₀+i*g₁} . . . }

Now substituting h0=g0+g1 yields:
SW_{i-k,j}+gⱼ=max{SW_{i-1,j}+h₀;g₁+max{SW_{i-k,j}+h₀+(k−1)*g₁;.g₁+SW_{1,j}+h₀+(j−1)*g₀} . . . }
And
SW_{i,j-k}+gᵢ=max{SW_{i,j-1}+h₀; g₁+max{SW_{i,j-k}+h₀+(k−1)*g₁;.g₁+SW_{i,1}+h₀+(i−1)*g₀)} . . . }

Now if we create variables GIₖ and GJₖ where
GIⱼ₋₁=max{SW_{i,j-1}+h₀;g₁+GIⱼ₋₂} and
GJᵢ₋₁=max{SW_{i-1,j}+h₀; g₁+GJᵢ₋₂} then
SW_{i-k,j}+gⱼ=GJᵢ₋₁; and SW_{i,j-k}+gᵢ=GIⱼ₋₁;

So the Smith Waterman equation can be recast as:
SW_{i,j}=max{SW_{i-1,j-1}+s(aᵢ,bⱼ); GJᵢ₋₁; GIⱼ₋₁; 0}

FIG. 2a still takes one cycle and has a long path, because at the beginning of the cycle the new Ax may have to propagate through only (210), (220) and (250), to create the new captured SWL (255), but the signal must continue on path (295) through (270) and (275) to the GI (280) and GJ (235) registers.

On the other hand FIG. 2b shows a processor constructed improving on the teachings of prior art. This is analogous to the processor taught in FIG. 1 with some added efficiency.

In the prior art, within a clock cycle, many of the subunits are used only a small fraction of the time, because all the terms in the Smith Waterman equation must be calculated. In the improvement, as per the prior art, the various circuit elements accept characters from one loaded character string, and one inputted character string, and over a number of clock cycles, make a comparison, bring in the other Smith-Waterman data from neighboring processors, determine a local maximum, and then pass the Smith-Waterman results along to the next processor. In the improvement, the calculation proceeds on the first term with the input of the Ax string element(200) and the comparison with the previously loaded value By (205). In one clock cycle these are compared and converted to a comparison value by the comp select function(210). The result is then added to the incoming SWL and checked for negative values (220), which is equivalent to the maximum of the first and last terms. At the same time the prior value of SWL plus h0 (260) is compared with the prior values of GI and GJ plus g1 (265). The maximum of the resulting sums (270, and 275) are both stored in the GI (280) and GJ (235) registers for the next cycle, and become the current GI (230) and GJ (225) values, the second and third terms, are being compared, the maximum of which is selected (240), which is then compared with the new SWL value (250). The maximum of this comparison is stored in the SWL register (255). On the next cycle the resulting registered SWL value (255) and its location from the counter (285) is compared with the previous maximum value and the maximum value and location are selected in the MOL function (290).

The calculations for one Smith-Waterman cell takes 2 cycles but the calculation for the basic Smith-Waterman equation takes only one cycle such that a new set of cell calculations can begin on each cycle. This simple two stage pipe gives a significant efficiency to this approach, over the prior art, since the original three additions (220), (270), and (275), one maxs function (220) and four maximum functions (270),(275),(240) and (250) in FIG. 2a have been rearranged in FIG. 2b so that the critical path has been reduced to one addition, a sign select and a maximum calculation, compared to two additional maximum functions in the prior art. (In this drawing, the double lined boxes are registers, and the dashed double lined boxes are static latches.)

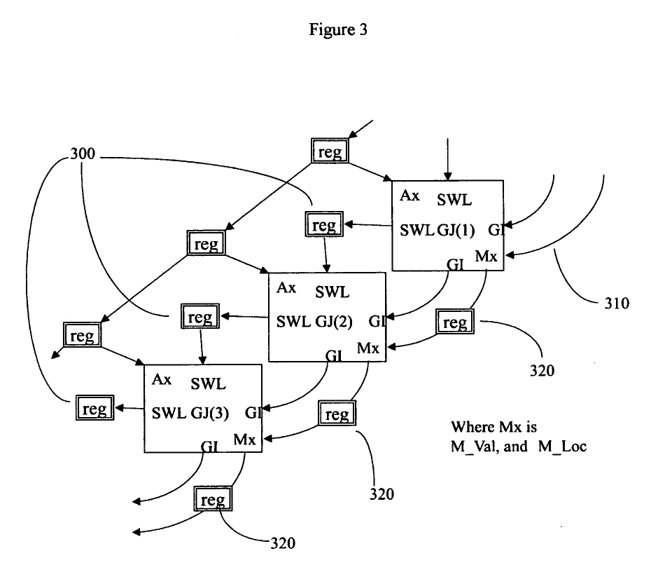

FIG. 3 shows how a plurality of processors interconnects on an integrated circuit chip, following the teaching of prior art. Registers hold information for one clock cycle until the processor has completed one full Smith-Waterman computation, and then pass information along to the next processor. The extra register between the SWL output and input (300) is needed to stage the diagonal calculation properly, as taught in the prior art. The Mx value (310) is the concatenation of the location of the maximum SWL value and the value itself It is necessary update and carry the cell location with the maximum SWL value to determine where the best match occurred. The GJ values stay within each processor, as they are the calculation of the row values. The counter/n (285) in FIG. 2 is the current location, which is concatenated with the SWL in the +Sign Select function (220) through the Max/sel function (250) on to the MOL function (290). The MOL function (290) compares the current and past maximum SWL values and passes the maximum value and its location on as the next Mx value. The maximum SWL value is at the end of the sub-string where the best match occurred. Following the comparison it is necessary to run the comparison in reverse to find the origin of the best match. This may be done using the comparison hardware, but since the strings are relatively short, it may also be done in a separate processor.

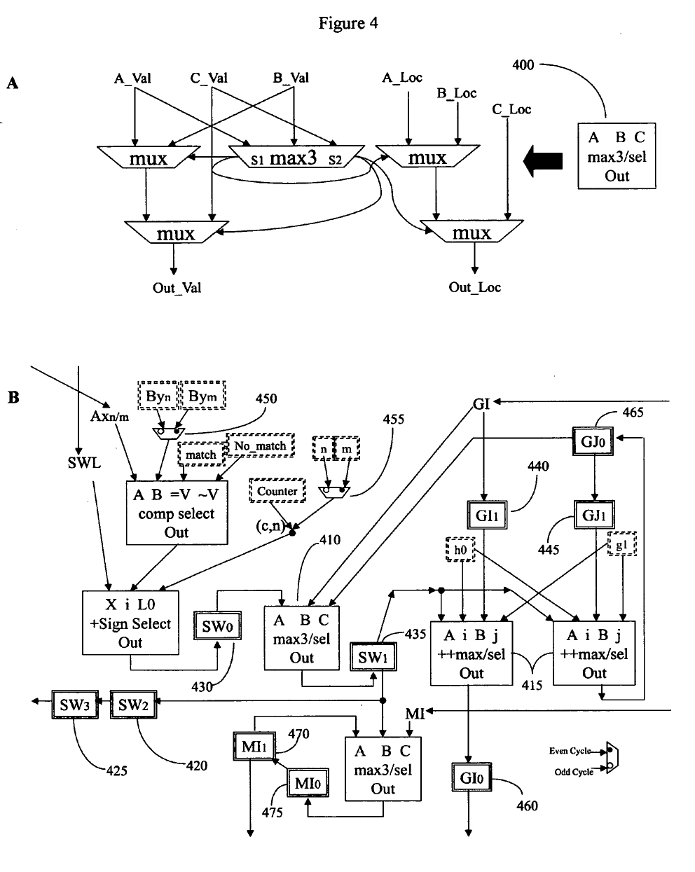

FIG. 4: A diagram showing an improved processor circuit. Here, the improved processor utilizes its components more efficiently by starting a second comparison operation executing before the first comparison operation is complete. By adding more registers, in the middle of operations previously done on one cycle, the clock frequency can be increased. The longest path in the FIG. 2 was from the comparison (210) to the SWL register (255). In the improved version the SWL computation path is split into two cycles, which allows the improved processor to correspondingly increase the clock frequency. Furthermore, the two stage two way maximum/selection operations (240, and 250) in FIG. 2 have been replaced with a 3 way maximum/selection operation (410) in the second stage of the SWL calculation, to balance the next critical path of GI and GJ contributions to SWL. This operation is only slightly longer than a two-way comparison. As can be seen in the associated drawing of max3/sel (400) in FIG. 4. Looking back at the original one cycle version in FIG. 2a the GI (280) and GJ (235) registers were placed after their ++max/sel functions (270) and (275) to ensure the GI and GJ values that were calculated on this cycle, were ready for the next calculation. Where as in FIG. 2b they were moved to before the next calculation to allow the previous SWL value in (255) to be used in the cycle that creates the next SWL value. In FIG. 4, both placements of the GI and GJ registers exist. GI₀ (460) and GJ₀ (465) are placed before the next calculation to capture the calculation from the odd cycle, and GI₁ (440) and GJ₁ (445) are placed before the max3/sel functions (415) to provide, along with the value in SW₁ (435) the proper values for the calculation. This means, at the same time, a totally separate operation even cycle operation from SW₀ (430), GI₀ (460) and GJ₀ (465) is completing a separate SW calculation through the max3/sel function (410), to be stored in SW₁ (435). The addition of the other registers (470), and (425) over those in (300) and (320) shown in FIG. 3, are added to align the two separate calculations.

Now each basic Smith-Waterman calculation takes 2 clock cycles to pass it's information on to the next engine, so the alternate cycle can be used to complete another unassociated operation. By interleaving the pipeline, all units in the diagram in FIG. 4 are productive on every clock cycle. This results in a somewhat larger dedicated processor than the diagram in FIG. 2b, but it can do twice as much in a shorter clock cycle.

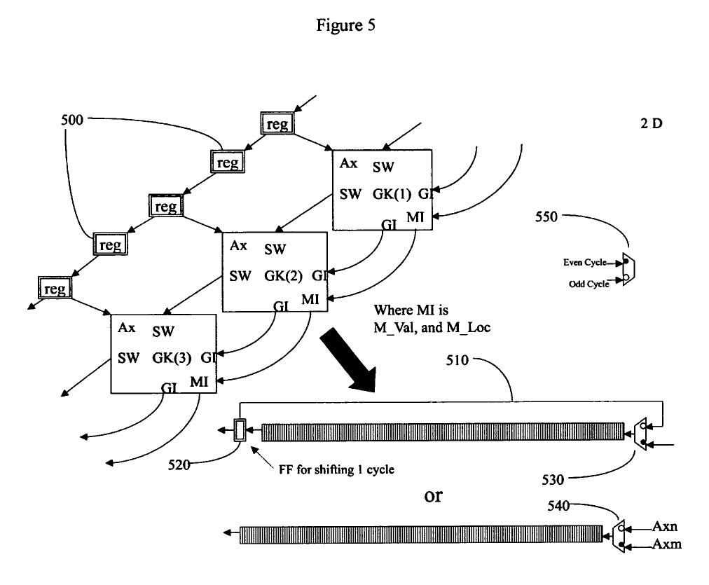

FIG. 5 shows how a plurality of improved processors interconnect on an integrated circuit chip. Registers SW2 (420) and SW3 (425), shown in FIG. 4, hold the SW value for the next processor, which eliminates the need for the intervening register (300), between processors, shown in FIG. 3.

In the improved processors, two independent calculations can occur one cycle removed from each other. This is done by interleaving the separate values of Ax, which requires the additional staging registers (500) as compared to FIG. 3 above. Thus the computational string cycles back through a second time. Essentially the processor's connection architecture folds back on itself (510).

As a result the improved processors are smaller and faster than prior art. Processor size (in terms of transistor or gate count) per unit of processing capability is effectively decreased because the interleaving circuitry. This effectively doubles output, requires only the addition of a few additional registers (employing a small number of transistors or gates) to the non-interleaved circuit of prior art.

In the design shown here, each processor can handle two separate interleaved computations, with each computation taking two clock cycles to execute. Every function is active on every cycle, and less logic is executed between cycles. This is effectively one computation per clock cycle with clock cycles that are at least 3 times faster than prior art, which more than offsets the additional register logic.

FIG. 6 shows how the efficiency of processor component utilization can be improved by using interleaving techniques. By adding the additional registers (420,425,430,475,440, 445, and 500) shown in FIGS. 4 and 5 and the additional mux switches (450, 455, and 530), the improved processor can handle two separate operations simultaneously. Here there are 3 cycles in each operation, with two cycles between each operation and the next logical operation N->N+1 or M->M+1. The components that are not used every other cycle above are used for a totally separate cell calculation. To minimize the top-level logic, one additional clock cycle is added at the end of the string (520) to shift the contents from the N,N+1 calculations to the M,M+1 calculations.

It should be noted here that the N,N+1 and M,M+1 calculations are entirely independent. That is to say the string comprised of Axm elements could be completely separate from the string comprised of Axn elements, and the string comprised of Bym and Byn elements could also be entirely separate strings. By eliminating or disabling the latch (520) and wire (510) in FIG. 5, two entirely separate comparisons may be done. The comparisons are interleaved between odd and even clock cycles as shown by the multiplexor key (550) in FIG. 5. If such multiplexors (450 and 455) in FIG. 4, were eliminated or disabled then a single string comprised of By elements would be compared with two strings comprised of Axn and Axm elements. Similarly, if the multiplexor (540) in FIG. 5 were also eliminated then a single string of Ax elements could be compared to two strings of Byn and Bym elements. As a result this architecture provides for a variety of two independent two-way comparisons, interleaved one cycle apart.

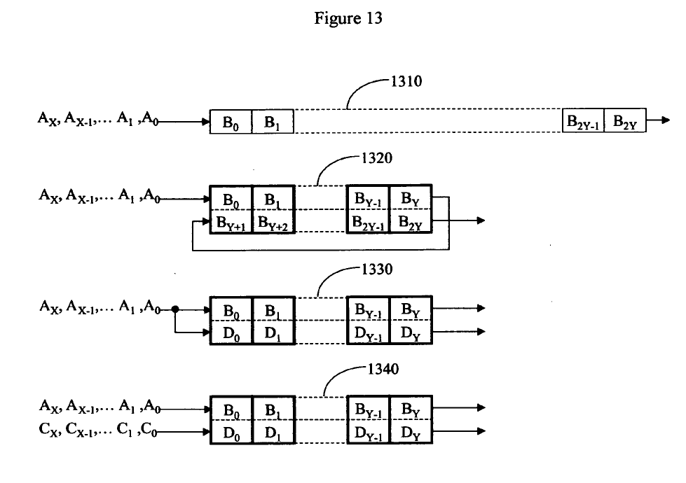

FIG. 13 is an abstraction of such non-interleaved and interleaved functions. A non-interleaved (1310) function would require 2Y elements to function the same as a folded interleaved function (1320). The folded interleaved function would only require one extra clock cycle to complete over the non-interleaved version, but would require only ½ the computational elements. Furthermore the folded interleaved function allows the option of two comparisons B and D with A (1330) or two separate comparisons A with B and C with D (1340), each of which only takes one more clock cycle than a single comparison. While this is the same conclusion reached by Borah, the improved embodiment described in this patent allows multiple independent comparisons without a common string, and multiple comparisons are performed on the same number of processors as a larger single comparison.

In addition to enabling faster and more economical 2 D bioinformatic systems to be constructed, the processor optimization techniques taught herein enable the construction of novel types of specialized processors dedicated to higher dimensional sequence (three or more) analysis systems.

As previously discussed, improved methods of doing higher dimensional sequence comparisons between three or more target sequences with unknown homology are highly desirable because of the nature of biological DNA sequence divergence. A single ancestral DNA sequence will typically, after genetic divergence into different organisms, undergo mutations, insertions, or deletions in different places. By making use of consensus logic (“two out of three” algorithms, etc.), faint low-homology matches between distantly related DNA sequences may be discovered that might otherwise be missed using conventional multiple 2 D approaches, or lossy higher dimensional approaches.

An example of how three-dimensional sequence comparisons can find relationships that might be otherwise missed by multiple two-dimensional sequence comparisons is shown below:

Assume that the sequence was originally a text message consisting of:
“This is the original text sent”

With time, transmission errors, misunderstanding, or other error source, this original text then mutates to three different alternate forms. In this example, these forms are:

1: This is not the original text sent
2: This is the original french text transmitted
3: This is the original letter sent

These three alternate forms would match-up as:

1: This is not the original text sent
2: This is the original french text transmitted
3: This is the original letter sent

If only sequences 1-2 were compared, the best possible match would be:

```
  1: This is not the original text sent
  2: This is the original french text transmitted
12: This is XXX the original XXXXXX text XXXXXXXXXXX
```

Here the XXX's represent confusion: Because version 1 has “not” and version 2 has “french” and “transmitted”, there is no easy way to tell what the original text was.

If only sequences 2-3 were compared, the best possible match would be:

```
  2: This is the original french text transmitted
  3: This is the original letter sent
23: This is the original XXXXXXXXXXXXXXXXXXXXXXX
```

Again the XXX's represent confusion. Version 2 has “french text transmitted”, and version 3 has “letter”. With only two sequences to compare, it is impossible to resolve which sequence has the correct information.

If only sequences 1-3 were compared, the best possible match would be:

```
  1: This is not the original text sent
  3: This is the original letter sent
13: This is XXX the original XXXXXXXXXXX sent
```

The best consensus would be:

123: This is XXX the original XXXXXXXXXXX sent

There is confusion as to how long the original message was, if it contained some sort of extra text between “is” and “the”, and the word “letter” would be rejected as not being part of the original message.

By contrast, when all three comparisions 1-2-3 are done at once, the results are:

1: This is not the original text sent
2: This is the original french text transmitted
3: This is the original letter sent
+: This is --- the original ------ text sent

If a 2 out of 3 consensus function is used, then the consensus would be:
3:3 “This”
3:3 “is”
2:3 “not” was not part of the original text
3:3 “the”
3:3 “original”
0:3 total disagreement, so neither “french” nor “letter” was part of the original text
2:3 “text”
2:3 “sent”

And the original “This is the original text sent” message” can be recreated.

FIG. 7 shows a diagram showing the relationships between the various cells required to do a simultaneous analysis of three different input character strings or arrays, such as three different nucleic acid sequences. The left side (700) shows that for a 3 D generalized Smith-Waterman type calculation, a processor calculating any given cell location will use values from processors computing the values for nearby cell locations in all three dimensions. The right side (710) illustrates graphically that upon projection back to two dimensions, the 3 D generalized Smith-Waterman type cell analysis gives results equivalent to the standard 2 D Smith-Waterman type analysis. That is, if for any one dimension, if all results for that particular dimension are “don't care” (weight=0), then the results for the remaining two dimensions will be equivalent to a standard 2 D Smith-Waterman type analysis.

Here a given match between any three characters from the three input character strings is compared to the previous I−1, J−1, K−1 match between the three strings, as well as other combinations, each representing a different possible combination of insertion or deletion events between the three strings. This is done according to the formula:
SW[i,j,t]=max{SW[i−1,j−1,t−1]+s(a[i],b[j],c[t]); SW[i−k,j,t]+g[I]; SW[i,j−k,t]+g[j]; SW[i,j,t−k]+g[t]; 0}
i is the sequence index in sequence 1
j is the sequence index in sequence 2
t is the sequence index in the test sequence
k is a variable length gap

One difference between the new higher dimensional string comparison processors taught here, and previous two dimensional string comparison processors, is that the criterion for a match between given string characters is not necessarily a simple: Does character[I]=character [j]? test. Rather, it is often preferable to use a “two out of three or three out of three” consensus logic function. Many different consensus functions are possible, some examples are:

Assume string1 is given weighting factor a, string2 is given weighting factor b, and string 3 is given weighting factor c, then an exemplary consensus function “Cx”, used for computing the various SW components is:

$$\begin{matrix}Ca [ I , j , k ] = a + b ( If Sting 1 [ i ] = String 2 [ j ] ) ; or \\ a + c ( If String 1 [ i ] = String 3 [ k ] ) ; or \\ b + c ( If String 2 [ j ] = String 3 [ k ] ) ; or \\ a + b + c ( if String 1 [ i ] = string 2 [ j ] = string 3 [ k ] ) or \\ 0 ( If String 1 [ i ] < > String 2 [ j ] < > String 3 [ k ] )\end{matrix}$$

or alternatively:

$$\begin{matrix}Cb [ I , j , k ] = a * b ( If Sting 1 [ i ] = String 2 [ j ] ) ; or \\ a * c ( If String 1 [ i ] = String 3 [ k ] ) ; or \\ b * c ( If String 2 [ j ] = String 3 [ k ] ) ; or \\ a * b * c ( if String 1 [ i ] = string 2 [ j ] = string 3 [ k ] ) or \\ 0 ( if String 1 [ i ] < > String 2 [ j ] < > String 3 [ k ] )\end{matrix}$$

Many other types of consensus functions are possible. By giving “partial credit” for “two out of three matches”, and greater credit for “three out of three matches”, etc., three and higher dimensional comparisons may uncover faint similarities that two-dimensional analysis may miss. If more then three input arrays are being compared, the consensus function will be altered accordingly (e.g. “three out of four”, etc.). Here these consensus functions will be designated as “two out of three or three out of three” functions, but it should be understood that this designation would apply to consensus functions for more than three input arrays as well.

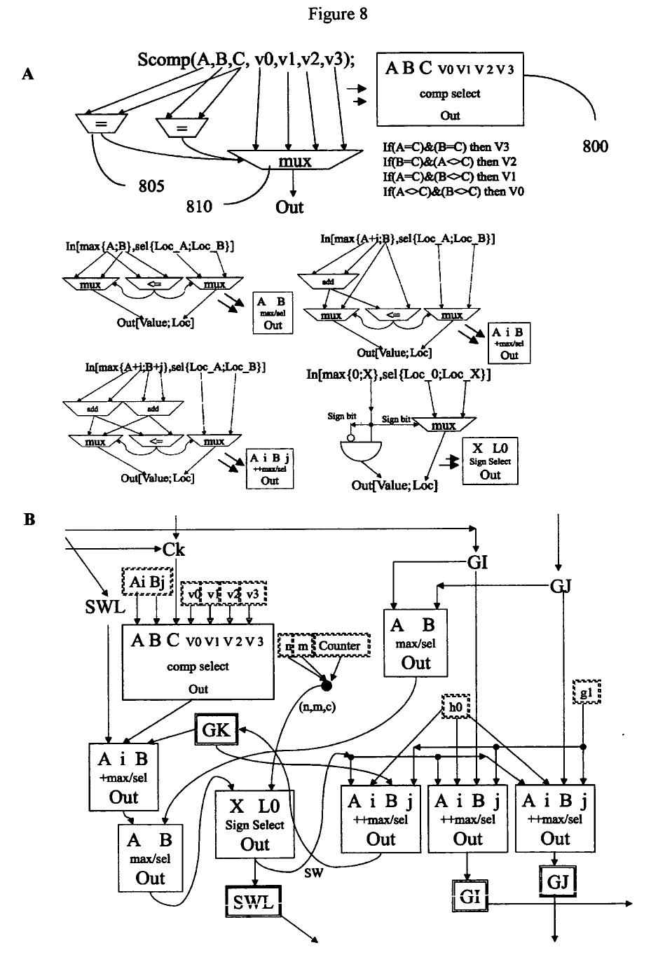

FIG. 8 shows an example of a processor optimized to perform three-dimensional comparisons between strings (arrays), using the higher dimensional form of the Smith-Waterman algorithm shown in FIG. 7 and described above. In particular the consensus function is implemented in comp select (800) block of logic. This implementation performs the following operation:

$$\begin{matrix}C [ I , j , k ] = v0 ( if String 1 [ i ] < > String 3 [ k ] and String 2 [ j ] < > String 3 [ k ] ) ; or \\ v1 ( If String 1 [ i ] = String 3 [ k ] and String 2 [ i ] < > String 3 [ k ] ) ; or \\ v2 ( If String 2 [ j ] = String 3 [ k ] and String 1 [ i ] < > String 3 [ k ] ) ; or \\ v3 ( if String 1 [ i ] = String 3 [ k ] and String 2 [ j ] = String 3 [ k ] ) \end{matrix}$$

which for simplicity does not implement all possible versions of the consensus functions described above, but may be easily expanded by adding another compare function (805) between A and B, and expanding the multiplexor (810) to select between 8 values instead of 4, with three of the values v3,v5,and v6 being unused since they are:
v3 (If String2[j]=String3[k] and String1[i]=String3[k] and String1[i]<>String2[j]),
v5 (If String1[i]=String3[k] and String2[i]° String3[k] and String1[i]=String2[j]), and
v6 (If String2[j]=String3[k] and String1[i]<>String3[k] and String1[i]=String2[j]),
   which are not mathematically possible. The function would then perform the following operation:
C[I,j,k]=
v0 (if String1[i]<>String3[k] and String2[j]° String3[k] and String1[i]<>String2[j]); or
v1 (If String1[i]=String3[k] and String2[i]<>String3[k] and String1[i]<String2[j]); or
v2 (If String2[j]=String3[k] and String1[i]° String3[k] and String1[i]<>String2[j]); or
v4 (if String1[i]<>String3[k] and String2[j]<>String3[k] and String1[i]=String2[j]); or
v7 (if String1[i]=String3[k] and String2[j]=String3[k] and String1[i]=String2[j]),
   which by the proper selection of v0,v1v2,v4, and v7, can perform any consensus function, in particular by setting: v0=0,v1=a+c,v2=b+c,v4=a+b, and v7=a+b+c, or v0=0,v1=a*c,v2=b*c,v4=a*b, and v7=a*b*c, consensus functions Ca and Cb above may be implemented.

Just as it is possible to implement a two-dimensional Smith-Waterman analysis in a one-dimensional array of processors by computing along the diagonal of a two dimensional matrix, so it is possible to implement a three dimensional Smith-Waterman style analysis in a two-dimensional array of processors, by computing along a planar cut through the three dimensional array.

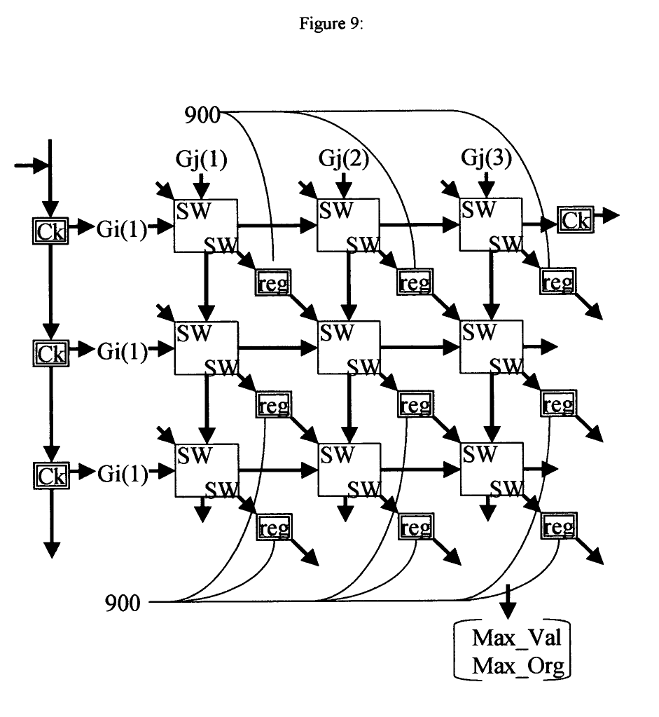

FIG. 9 shows how a plurality of three-dimensional string comparison processors can interconnect on an integrated circuit chip.

As for the two dimensional case, the three dimensional processors can either complete their 3 D Smith-Waterman cell computations, and pass the results along in pairs of registers(900), or also use interleaving techniques to perform multiple 3 D Smith-Waterman cell computations simultaneously.

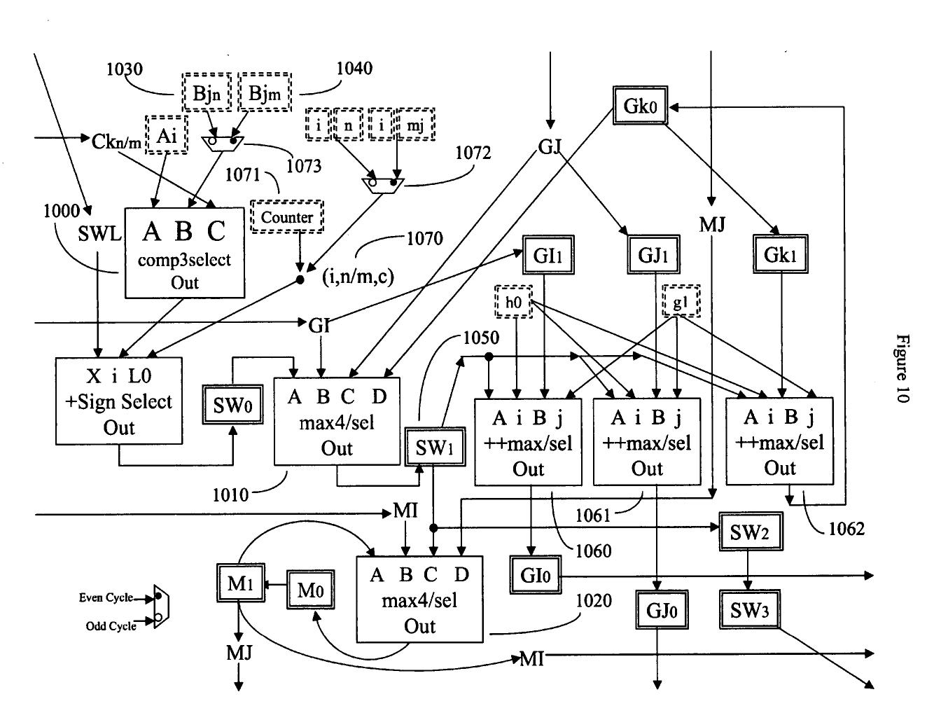

FIG. 10 is a diagram of an interleaved version, in the same manner as FIG. 4, of the three-dimensional processor shown in FIG. 8. This has the same set of functions as FIG. 4, but includes the third dimension shown logically in FIG. 8. There are two different functions; comp3select (1000) and max4/sel (1010, and 1020) than were shown in FIG. 8. Similar to FIG. 4, this interleaved processor produces an SW result every 2 cycles and can alternate processing two different comparisons signified by Bjn (1030) and Bjm (1040) above. In a similar fashion to FIG. 4, all the external registers have been included in this diagram. The diagram of operation of this embodiment of an interleaved three-dimensional processor is the same as shown in FIG. 2, but for three dimensions instead of two. On the first cycle the three strings are compared, on the second cycle the maximum of all four elements is performed to output SW1 (1050), the calculated value of the cell. On the next cycle the three column, row and height (GI, GJ, and GK) coefficients are updated, along with the maximum (1060,1061, and 1062). The three-dimensional location is shown as a 3-tuple [i,n/m,count] (1070). The count increments every two cycles (1071). Every other cycle n or m are alternatively chosen for the current location of the cell be processed (1072), as well as the By element to be used in the comparison (1073)

The comp3select (1100) function in FIG. 11 has 4 separate values for each possible combinations of comparing the three strings. The max4 (1110) function shows a single bus bit's maximum logic. The AND/OR/MUX (1120) logic is repeated for each bit in the words A through D, and is connected from the highest order to the lowest order bit. If a strict maximum occurs only one of the Sa through Sd bits is still high at the lowest order bit comparison. The resulting Sa-Sd are encoded (1130) for use in the max4/sel function. In a manner similar to Addition, look-ahead logic may be added to minimize the number of gate levels in real operation.

FIG. 12 shows the interconnection of multiple interleaved, three-dimensional processors. In a manner similar to FIG. 9, the processors are connected into a two dimensional array. Processing begins on every other cycle until the results are passed out of the last processors on the right. The results are then fed into a single register for Gi and a single register for SW (1200) and fed back into the array to be processed on the second of every two cycles. This single register shifts the results from even operation to odd. In the example above the engine should be executed for a total of 24 cycles. The maximum value of all the cells calculated and the origin of the C string for that cell are available on the outputs (1210) two cycles after the last calculated SW value is calculated.

Note the interleaving is only in one direction. Interleaving in more than one direction requires more clock cycles of operation, and hence more registers. For example, to interleave in two directions requires 4 clock cycles per operation, with each quadrant of the unfolded calculations taking on clock cycle. This would require 4 additional registers for SW and two additional registers for all the other operations. In general, given sufficient registers any amount of folding may be done in either direction, but this also increases the size of each processing element, which suggests a tradeoff exists between the amounts of folding versus the number of processing elements that can fit on one chip.

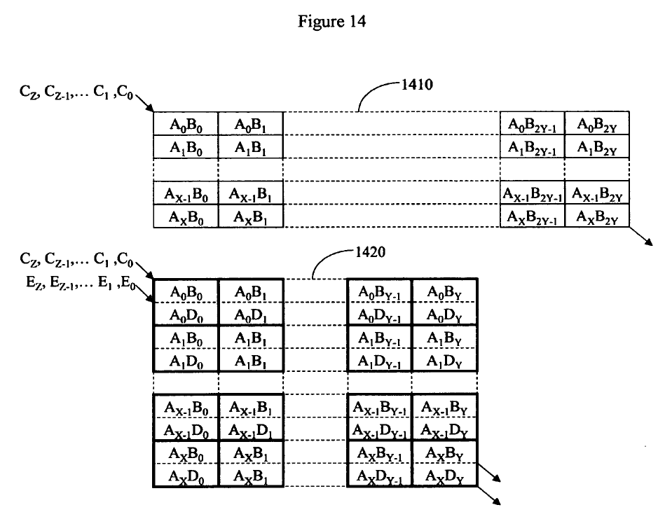

Still by building in the folding, multiple comparisons are possible. FIG. 14 shows an abstraction of three-dimensional non-interleaved and interleaved functions. The non-interleaved function compares Strings A, B and C, where C can be any length but A and B are bounded by the number of elements in the chip 1410. Clearly by folding B half the elements can do the same comparison in half the processing elements as in the two dimensional case, but such folded interleaving also allows strings A,B and C to be compared at the same time strings A,D and E are compared 1420, with the restriction that D and B are each ½ the size of the folded B in the single three-way comparison case. The same calculations as were done to recast the 2 dimensional Smith-Waterman equations can apply to N dimensions. Rotating the dimensions leads to the following:
SWi . . . n=max{SWi−1 . . . n−1+s(ai,bj . . . fn); GIi−1; . . . GNn−1; 0}

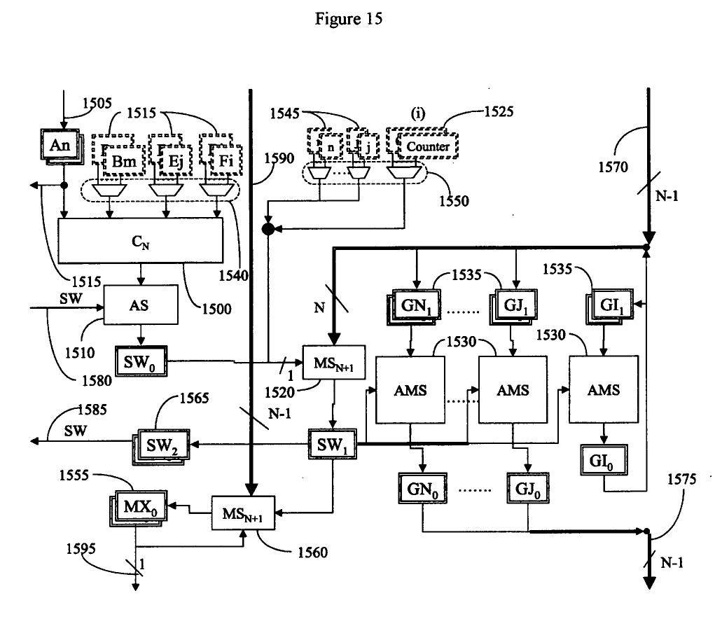

Clearly, by extrapolation, since a 2 dimensional Smith-Waterman comparison can be executed in a one dimensional array of processors, and a 3 dimensional Smith-Waterman comparison can be executed in a 2 dimensional array of processors, the N dimensional equation shown above can be executed in an N−1 dimensional array of processors, each of which would have the structure shown in FIG. 15.

Where the functional blocks (1500), (1510), (1520), and (1530), respectively, have the following functions:

```
Function C_{N}(X₀,X₁,...X_{N})
    K=0;
    For I = 0 to N;
        For J=I+1 to N;
          A_{K} = (X_{I}=X_{J}); /* The Kth bit of A is
          set with bit value for (XI=XJ) */
          K = K+1;
        Next J;
    Next I;
    CN = Values(A);
end CN;
Function AS(X,V);
    AS=X+V;
    If AS < 0 then AS = 0;
end AS;
Function MSN(VL0,VL1...VLN as [V;L]) as [V;L]
    V = V0;
    L = L0;
    For I=1 to N;
        If VLI.V>V then
            V = VLI.V;
            L = VLI.L;
        End if;
    Next I;
    MS.V = V;
    MS.L=L;
end MSN;
Function AMS(VL0,VL1 as [V;L]) as [V;L]
    V0 = VL.V0+H0;
    VI = VLI.V+G1;
    If V0>V1 Then
        AMS.V = V0;
        AMS.L=VL.L0;
    Else
        AMS.V = V1;
        AMS.L =VL.L1;
    end if;
end AMS;
```

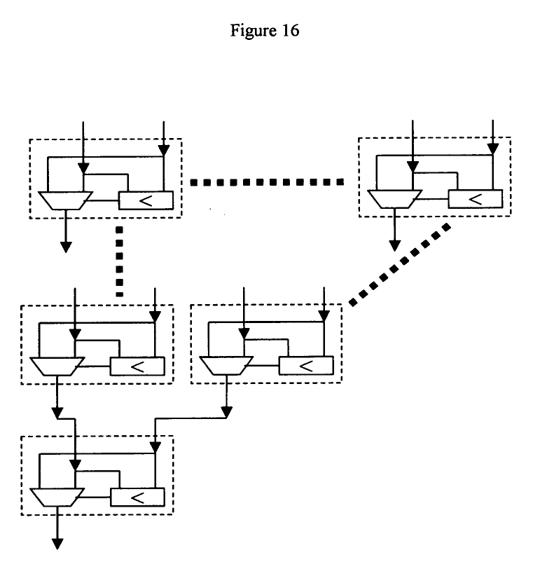

The Comparison function uses an array of Values of size 2 K where K=N!/(N-2)!*2! for the match coefficients. For large N this array can get quite large, but since all processors have the same array contents, the copies of the array may be shared between multiple processors, providing an individual read port exists for each processor. The MSN and AMS functions have input parameters VL, which are a structure consisting of two numbers; a value V, and a location L. As shown above, the MSN function performs an N way maximum calculation, which can be done in parallel with a tree of N−1 two way comparisons driving N−1 two way selects, in a log2(N) tree structure shown in FIG. 16.

Such an N dimensional processor would consist of one Cn function (1500), one AS function (1510), two MSN functions (1520) and (1550), and N AMS functions (1530). The location of each processor is determined by an N-tuple of (i,j . . . n), where the i is determined by a selected counter.

Each N dimensional processor must be connected into an N−1 dimension array of processors. The N−1 G inputs(1570) and the N−1 MX inputs (1590) come from the (j−1,k . . . n), (j,k−1 . . . n) . . . (j,k . . . n−1) processors on each N−1 line of prior processors. The Mx output (1590) fans out to the N−1 subsequent processors in locations (j+1,k . . . n), (j,k+1 . . . n) . . . (j,k . . . n+1). The N−1 G outputs also connect to these processors, GJ to (j+1,j . . . n), GK to (j,k+1 . . . n), . . . . GN to (j,k . . . n+1). The An string elements are distributed down the diagonal of the N−1 dimensional space, starting at the (0,0 . . . 0) processor and fanning out to the (1,0 . . . 0),(0,1 . . . 0) . . . (0,0 . . . 1) processors. Since any processor would get the same An value from any of its preceding processors, only one axis is connected to the An input (1505) of each processor, and except for edge processors, the output (1515) fans out to only one processor. The SW input (1580) comes from the (j−1,k−1 . . . n−1) processor, and the SW output is sent to the (j+1,k+1 . . . n+1) processor.

An array of N dimensional processors can also handle M independent comparisons if there are sufficient registers added. For M independent comparisons, the register queues; Ai (1505), and MX (1555) and the mux selectable arrays; Bj through Fn (1515), the counter (1545), the coefficients j through n (1525) all must have M elements. The G value queues (1535) have M−1 elements because the 0th register is separate, and the SW queue (1565) has N*M−2 elements because the SW−1, . . . −1 element is (1,1 . . . 1) diagonally away from where it is next used, which requires N steps to reach, for each of the M comparisons. For large values of N and M these queues could get quite large, but in these cases the area may be reduced by using memories rather than individual registers.

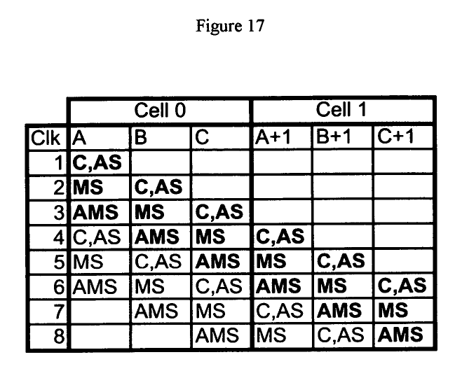

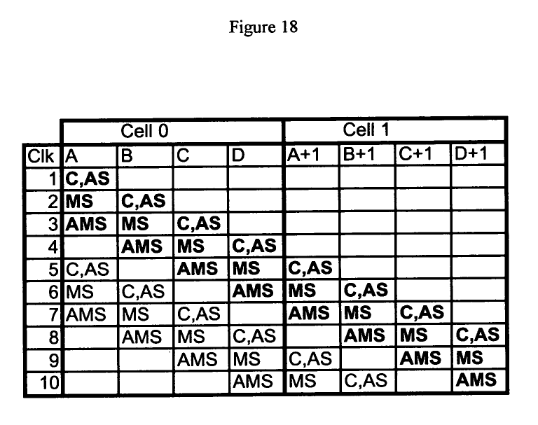

With the proper selection on the Muxes (1540) and (1550) on the proper clock cycle, the different comparisons and concatenations of the corresponding coordinates can occur in an interleaved. FIGS. 17 and 18 show the operations that occur on a portion of the clock cycles for M=3 and M=4, for two adjacent cells. The CN (1500) and AS (1510) functions are listed together in one cycle, because there is no register separating them. The MSN function (1520) occurs on the next cycle, and the AMS functions (1530) execute on the third cycle. For brevity, the MSN function (1560), is not listed in these charts, but occurs simultaneous with the execution of the AMS functions (1530).

In the case shown in FIG. 17, there are three comparisons; A, B, and C, which are occurring in two cells; 0 and 1. On the beginning of clock cycle 4, the results of comparison A from cell 0 are available to begin processing the next cycle of comparison A, called A+1, on cell 1. Of course simultaneously the cell 0 is beginning its next comparison calculation for A. This requires the muxes (1540) and (1550) select between three different sets of strings and locations, respectively. The counters (1525) increment after the cycle they have been selected on. As can be seen by this chart, there are no cycles, which have no activity for any of the comparisons, because the number of simultaneous comparisons is the same as the depth of the pipeline, where as in FIG. 16, four pipelined comparisons occur, which requires four cycles to free up the C,AS functions in each cell, at which time the next element in that comparison can begin. This case requires cycling through four clock counts on each processor.

Regardless of the dimension or number of parallel comparisons, prior to beginning the comparisons, the G (1535), MX (1555) and SW (1565) queues must be cleared, the counters (1525) must be reset, and the appropriate strings must be loaded such that each of their elements reside in the proper array locations (1515) in each of the processors, one string element from each string, into each processor in the array. These loaded strings remain static throughout the subsequent comparisons. For M parallel comparisons, when clocking begins, the M inputted strings must be shifted into the array of processors in an interleaved fashion, such that the next element from the Tth string is inputted every Tth clock cycle. For M strings of length N, after N+D+2 cycles, where D is the sum of the dimensions of the processor array, and for the next M cycles, the M maximum Smith-Waterman scores and locations of those scores for the M comparisons, are outputted from the array. In this fashion, using an N−1 dimensional array of processors, M parallel independent N-Dimensional comparisons may be accomplished faster than other prior art alternatives.

1. An electronic circuit for performing lossless similarity analysis between three or more input linear character arrays, said circuit being divided into a plurality of processors, each processor performing the operations of:
retrieving one character from each of the various input arrays;
comparing the various characters using a consensus function between three or more input characters;
adding the results of the comparison to a function based on the comparison results obtained from neighboring subunits that are analyzing different regions of the various input arrays;
and determining the maximum of such inputs from neighboring subunits;
wherein inputs to the electronic circuit include data from the various input arrays, and outputs from the electronic circuit include comparison data and pointers to regions of higher homology between three or more of the input arrays.

2. The electronic circuit of claim 1, wherein the input arrays contain biological nucleic acid sequence data, and the comparison between the input arrays is a multi-dimensional generalization of the Smith-Waterman type analysis.

3. The electronic circuit of claim 1, wherein said consensus function between three or more-of the characters is one of partial credit and greater credit consensus functions.

4. The processor of claim 1, wherein the processor interleaves results from a neighboring processor of the same type during each comparison analysis so as to perform multiple comparison operations during at least some of the same clock cycles.

5. The processor of claim 1, wherein the processor includes means to interleave the calculation pipeline so that the logical units in the processor are productive on every clock cycle.

6. The processor of claim 1;
wherein said processor determines regions of similarity between input data consisting of three separate linear character arrays;
wherein said processor selects a single character from a different position from each of the three said input character arrays,
wherein said comparison function determines if said three selected characters have a 3 out of 3, 2 out of 3, 1 out of 3, or 0 out of 3 agreement.

7. The processor of claim 1;
wherein said processor determines regions of similarity between input data consisting of three separate linear character arrays;
wherein said processor selects a single character from a different position from each of the three said input character arrays,
wherein said comparison function determines if said three selected characters have a 3 out of 3, 2 out of 3, 1 out of 3, or 0 out of 3 agreement;
wherein said function based on comparison results is a three dimensional extension of the Smith-Waterman function to character arrays where each character requires 2 or more bits per character to represent.

8. The processor of claim 1;
wherein said processor determines regions of similarity between input data consisting of three separate linear character arrays;
wherein said processor selects a single character from a different position from each of the three said input character arrays,
wherein said comparison function determines if said three selected characters have a 3 out of 3, 2 out of 3, 1 out of 3, or 0 out of 3 agreement;
wherein said function based on comparison results is a three dimensional extension of the Smith-Waterman function to character arrays;
wherein said linear character arrays are linear character arrays of nucleic acids.

9. An improved processor, a plurality of which are organized into an array and embedded into an integrated circuit chip used to compare one or more loaded strings of characters, with one or more inputted strings of characters in a lossless manner;
wherein each improved processor performs the operations of:
receiving a prior comparison value and partial comparison values from one of a set of registers, and a prior of said improved processors;
receiving a character out of one of said one or more inputted strings of characters, said character being received from one of a chip input and a first of said improved processors;
comparing said received character with one character from each of said loaded strings of characters;
Generating a new comparison value and said partial comparison values;
Selecting the maximum of said prior and new comparison values; and
sending said received character, said maximum comparison values, and said partial comparison values to one of a chip output and one or more subsequent said improved processors;
wherein each improved processor starts a second set of said operations before a first set of said operations is complete.

10. The processor of claim 9, wherein the processor interleaves said operations such that each set of said operations is takes at least two clock cycles and one of each set of operations is completed each clock cycle.

11. The processor of claim 9, wherein the outputs from the processor includes comparison data and pointers to regions of highest comparison between the input strings, and the two or more loaded strings.

12. A chip as in claim 9 herein each of said inputted strings is compared with one or more of said loaded strings.

13. A chip as in claim 9 wherein each of said inputted strings is compared with at least one of said loaded strings, which is not compared with any other inputted string.

14. A chip as in claim 9, wherein said comparisons of each of said inputted strings with said loaded strings complete successively within one clock cycle of each other.

15. The processor of claim 9, wherein the means to interleave the operations includes using additional registers.

16. The processor of claim 9, wherein the inputted and loaded strings contain biological nucleic acid sequence data, and the comparison between the inputted and loaded strings is a multi-dimensional generalization of a Smith-Waterman type analysis.

17. The processor of claim 9, wherein the loaded strings contain text search data and the comparison is done against inputted strings of text information from search databases using a multi-dimensional generalization of a Smith-Waterman type analysis.

18. An improved processor, a plurality of which are organized into a two or more dimensional array and embedded into an integrated circuit chip used to perform multiple lossless similarity analyses between multiple groups of two or more loaded strings of characters, and one inputted string of characters.

19. An integrated circuit chip as in claim 18; wherein said multiplicity of independent similarity analyses are performed in parallel.

20. An integrated circuit chip as in claim 18; wherein said multiplicity of independent similarity analyses are performed in parallel, and the results of said analyses are obtained on successive clock cycles after completion of the first of said multiplicity of independent similarity analyses.
